(19) United States
(12) Patent Application Publication
(10) Pub. No.: US 2016/0045223 A1
(43) Pub. Date: Feb. 18, 2016

(54) INJECTOR FOR HAIR IMPLANT WITH AUTOMATIC FOLLICLE SUPPLY FUNCTION

(71) Applicant: Electronics and Telecommunications Research Institute, Daejeon (KR)

(72) Inventors: Kyu Hyung KIM, Daegu (KR); Tae Wuk BAE, Daegu (KR); Hyung Soo LEE, Daegu (KR); Yong Chul JUNG, Daegu (KR); Soo In LEE, Daejeon (KR); Chang Hyuk HONG, Daegu (KR)

(21) Appl. No.: 14/731,888

(22) Filed: Jun. 5, 2015

(30) Foreign Application Priority Data
Aug. 14, 2014 (KR) 10-2014-0105996

(51) Int. Cl. A61B 17/34

(52) U.S. Cl. CPC A61B 17/3468; A61F 2/10

(57) ABSTRACT

Disclosed is an injector for hair implant, and more particularly, to an injector for hair implant with an automatic follicle supply function which is capable of continuously and automatically supplying follicles so that the follicles are rapidly and easily implanted into a scalp.

### CROSS-REFERENCE TO RELATED APPLICATION

This application claims priority to and the benefit of Korean Patent Application No. 10-2014-0105996, filed on Aug. 14, 2014, the disclosure of which is incorporated herein by reference in its entirety.

### BACKGROUND

1. Field of the Invention

The present invention relates to an injector for hair implant, and more particularly, to an injector for hair implant with an automatic follicle supply function which is capable of continuously and automatically supplying follicles.

2. Discussion of Related Art

Generally, an injector is a device configured to implant hairs in a skin of a human body from which hairs are lost due to a disease or an accident.

The injector may be used to implant eyebrows, a beard, pubic hairs and hairs.

A conventional injector includes a needle in which a follicle is inserted, and a core (a rod member) which is slidably installed in the needle. The needle in which the follicle is received is inserted into a patient's scalp, and thus the follicle is implanted.

At this time, the typical injector includes one needle and one core installed in the needle, and thus only one strand of hair may be implanted at a time. Therefore, a time required in a hair implant is too long, and thus a procedure time is also long. And a patient has pain for a long period of time.

To solve the problems, there has been proposed a multi-channel injector which has two or more needles and cores.

However, in the case of the multi-channel injector, after the follicle is implanted, an operator should perform an operation in which a new follicle is put into the needle, and an operation time is increased, and inconvenience is involved.

### SUMMARY OF THE INVENTION

The present invention is directed to an injector for hair implant with an automatic follicle supply function which is capable of continuously and automatically supplying follicles so that the follicles are rapidly and easily implanted into a scalp.

According to an aspect of the present invention, there is provided an injector for hair implant with an automatic follicle supply function, including a main body; a follicle supply part coupled with the main body to supply a plurality of follicles; a needle disposed at a front side of the main body; a rod member installed at the main body to move forward the follicle supplied through the follicle supply part and disposed in the needle; a transfer part configured to move the follicle disposed at the follicle supply part to a front side of the rod member; and a rod driving part installed at the main body to linearly move the rod member, wherein the follicle supply part and the transfer part automatically supply a new follicle in front of the rod member, when one of the follicles is implanted into a scalp by the rod member.

A plurality of follicle supply pipes may be disposed in the follicle supply part to be supplied to the main body, and the follicles may be disposed in the follicle supply pipe, respectively.

The follicle supply part may include a fixed member formed in a cylindrical shape of which an outer circumferential surface is disposed in an up and down direction; a rotating member rotatably disposed at an inner side of the fixed member; and a rotation driving part configured to rotate the rotating member, and the plurality of follicle supply pipes may be installed at the rotating member to revolve around a rotating center point of the rotating member, when the rotating member is rotated.

A discharging port configured to discharge the follicle supply pipe installed at the rotating member to an outside of the follicle supply part may be formed at the fixed member in an up and down direction.

The transfer part may include an upper pressing part disposed above the discharging port; a lower pressing part disposed under the discharging port; and an up and down driving part configured to move upward and downward the upper pressing part and the lower pressing part, and the follicle supply pipe disposed at the discharging port may be separated from the follicle supply part by a downward movement of the upper and lower pressing parts due to the up and down driving part, and then disposed in front of the rod member.

The transfer part may move again a waste follicle supply pipe in which the follicle is used to the follicle supply part, and the rotation driving part may rotate the rotating member and may dispose a new follicle supply pipe at the discharging port.

The follicle supply part may include a housing having the plurality of the follicle supply pipes disposed therein in a row, and the discharging port formed at one end thereof; and a push member installed in the housing to push the follicle supply pipe toward the discharging port, and the transfer part may move the follicle supply pipe discharged through the discharging port by the push member to the front side of the rod member.

The transfer part may include an upper pressing part disposed at a front upper side of the discharging port; a lower pressing part disposed at a front lower side of the discharging port; and an up and down driving part configured to move upward and downward the upper pressing part and the lower pressing part, and the follicle supply pipe disposed between the upper pressing part and the lower pressing part may be disposed in front of the rod member by a downward movement of the upper and lower pressing parts due to the up and down driving part.

A lower spring may be installed at a lower surface of the lower pressing part to elastically support the lower pressing part in an upward direction in which the upper pressing part is disposed.

The discharging port may be closed when the upper pressing part is moved down.

The transfer part may move a waste follicle supply pipe, in which the follicle is used, in a lateral direction of the discharging port, and the push member may push the follicle supply pipe disposed in the housing toward the discharging port and thus may separate the waste follicle supply pipe from the transfer part.

The push member may be a pressing spring, and a stopper by which the waste follicle supply pipe disposed between the upper pressing part and the lower pressing part and horizontally moved by the elastic restoring force of the pressing spring is stopped may be formed at the main body, and the lower pressing part may support the waste follicle supply pipe caught and stopped by the stopper, and when the lower pressing part is moved down, the waste follicle supply pipe caught and stopped by the stopper may be moved down along with the lower pressing part and then moved to a collecting part.

An inclined surface inclined in a direction opposite to a supply direction of the new follicle supply pipe may be formed at a portion of an upper surface of the lower pressing part in which the waste follicle supply pipe is disposed, and when the lower pressing part is moved down, the waste follicle supply pipe disposed at an upper portion of the inclined surface may be moved to the collecting part along the inclined surface.

The needle may be fixed to a front side of the main body, and disposed to be on a line with the rod member, and the follicle supply pipe supplied to the main body by the transfer part may be disposed in a row between the rod member and the needle.

The follicle supply pipe may be configured with the needle, and a needle moving part which linearly moves the needle supplied from the follicle supply part and disposed in front of the rod member may be installed at the main body, and the needle may be disposed in front of the main body by the needle moving part.

The rod member may be installed at an inner side of the needle moving part to be linearly movable.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a structural view of an injector for hair implant according to a first embodiment of the present invention;

FIG. 2 is a structural view of a follicle supply part and a transfer part of the injector for hair implant according to the first embodiment of the present invention;

FIGS. 3A to 3D is a view illustrating an operation procedure of the follicle supply part and the transfer part of the injector for hair implant according to the first embodiment of the present invention;

FIGS. 4A to 4F is a view illustrating the operation procedure of the injector for hair implant according to the first embodiment of the present invention;

FIG. 5 is a structural view of a follicle supply part and a transfer part of an injector for hair implant according to a second embodiment of the present invention;

FIGS. 6A to 6F is a view illustrating an operation procedure of the injector for hair implant according to the second embodiment of the present invention;

FIG. 7 is a structural view of an injector for hair implant according to a third embodiment of the present invention;

FIG. 8 is a structural view of a follicle supply part and a transfer part of the injector for hair implant according to the third embodiment of the present invention;

FIGS. 9A to 9E is a view illustrating an operation procedure of the follicle supply part and the transfer part of the injector for hair implant according to the third embodiment of the present invention;

FIGS. 10A to 10F is a view illustrating the operation procedure of the injector for hair implant according to the third embodiment of the present invention;

FIG. 11 is a structural view of an injector for hair implant according to a fourth embodiment of the present invention; and

FIGS. 12A to 12F is a view illustrating an operation procedure of the injector for hair implant according to the fourth embodiment of the present invention.

### DETAILED DESCRIPTION OF EXEMPLARY EMBODIMENTS

### First Embodiment

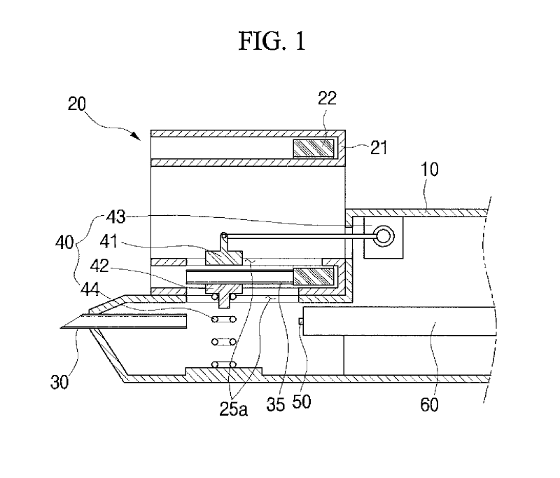

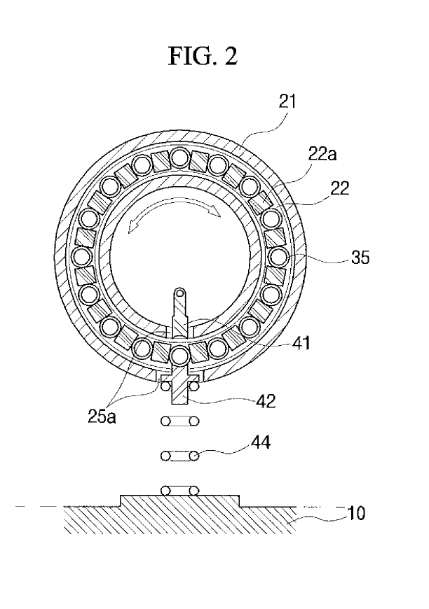

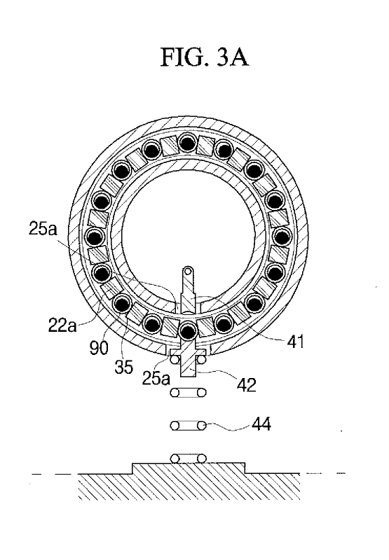

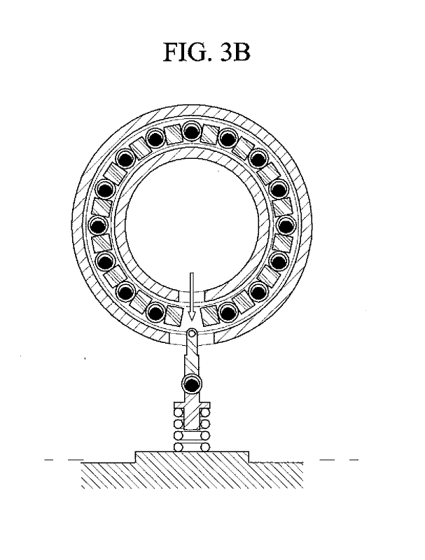

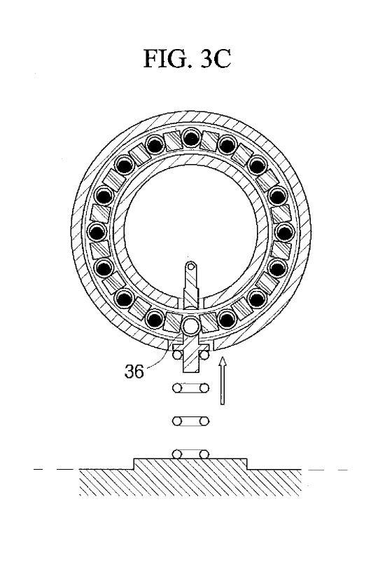

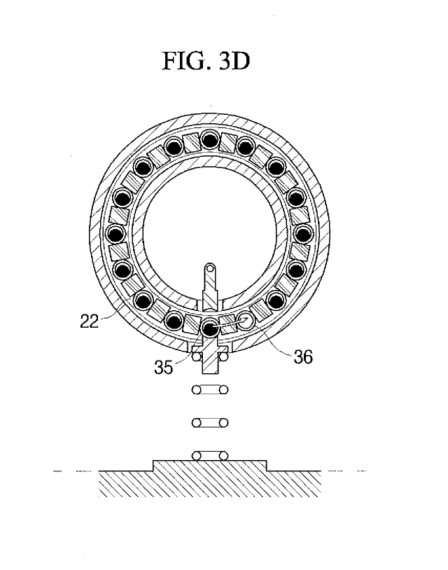

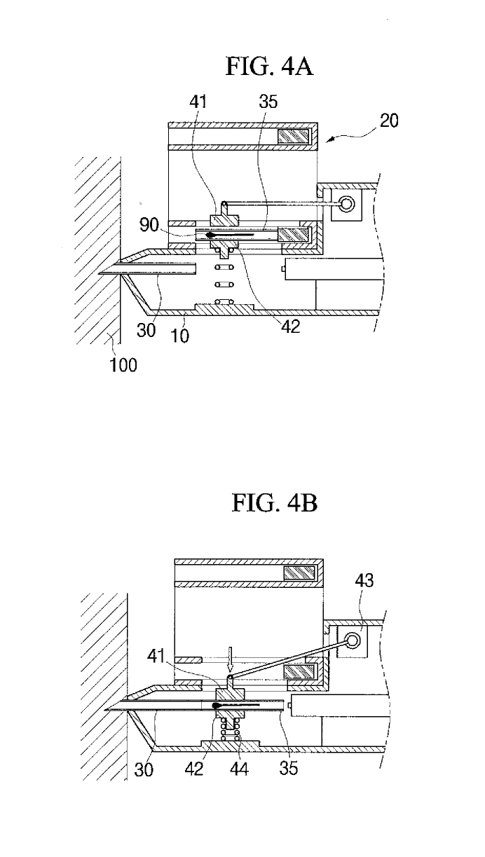

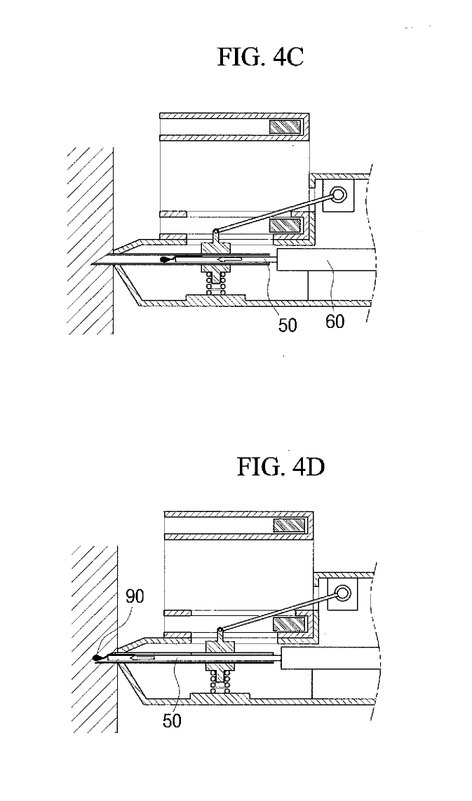

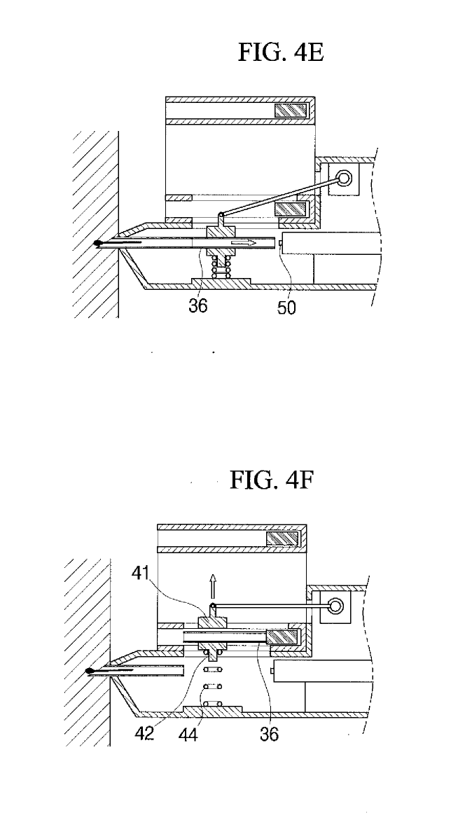

FIG. 1 is a structural view of an injector for hair implant according to a first embodiment of the present invention, FIG. 2 is a structural view of a follicle supply part and a transfer part of the injector for hair implant according to the first embodiment of the present invention, FIGS. 3A to 3D is a view illustrating an operation procedure of the follicle supply part and the transfer part of the injector for hair implant according to the first embodiment of the present invention, and FIGS. 4A to 4F is a view illustrating the operation procedure of the injector for hair implant according to the first embodiment of the present invention.

As illustrated in FIGS. 1 to 4, the injector for hair implant according to the present invention includes a main body 10, a follicle supply part 20, a needle 30, a transfer part 40, a rod member 50, and a rod driving part 60.

The main body 10 defines an entire exterior of the injector for hair implant of the present invention, and a shape thereof may be changed variously.

The follicle supply part 20 is coupled to the main body 10, and serves to supply a plurality of follicles 90.

The follicle supply part 20 may be fixedly coupled to the main body 10, or may be removably coupled to the main body 10.

A plurality of follicle supply pipes 35 are disposed in the follicle supply part 20, and a follicle 90 is inserted into each follicle supply pipe 35, and the follicle supply part 20 supplies the follicle supply pipes 35 to the main body 10.

In the embodiment, the follicle supply part 20 includes a fixed member 21, a rotating member 22, and a rotation driving part.

The fixed member 21 has a cylindrical shape, and is coupled to the main body 10 so that a circumferential surface thereof is disposed upward.

The rotating member 22 is rotatably disposed at an inner side of the fixed member 21.

The plurality of follicle supply pipes 35 are installed at the rotating member 22.

More specifically, as illustrated in FIG. 2, fixed protrusions 22a spaced from each other are formed around the rotating member 22, and the follicle supply pipes 35 are inserted and disposed between the fixed protrusions 22a and the fixed member 21.

The rotation driving part (not shown) configured with a motor or the like serves to rotate the rotating member 22.

By the follicle supply part 20 having the above-described structure, the follicle supply pipes 35 are rotated about a rotating center of the rotating member 22, when the rotation driving part rotates the rotating member 22.

The needle 30 is a part which is inserted into a scalp, and fixedly installed at a front portion of the main body 10.

The transfer part 40 serves to move the follicle 90 disposed in the follicle supply part 20 in front of the rod member 50.

To this end, a discharging port 25a which discharges the follicle supply pipe 35 installed at the rotating member 22 to an outside of the follicle supply part 20 is formed at the fixed member 21 in an up and down direction.

The transfer part 40 includes an upper pressing part 41, a lower pressing part 42, and an up and down driving part 43.

The upper pressing part 41 is disposed above the discharging port 25a, and a concave groove corresponding to a shape of the follicle supply pipe 35 is formed at a lower surface thereof.

The lower pressing part 42 is disposed under the discharging port 25a, and a concave groove corresponding to the shape of the follicle supply pipe 35 is formed at an upper surface thereof.

The up and down driving part 43 serves to move the upper pressing part 41 and the lower pressing part 42 up and down, and may have various conventional structures.

Due to the transfer part 40, the follicle supply pipe 35 may be separated from the follicle supply part 20 by a downward movement of the upper pressing part 41 and the lower pressing part 42 by the up and down driving part 43, while being disposed in the discharging port 25a, and then may be disposed in front of the rod member 50.

Of course, the transfer part 40 may move the follicle supply pipe 35 downward and then upward again.

A lower spring 44 is installed at a lower surface of the lower pressing part 42 to elastically support the lower pressing part 42 in an upward direction in which the upper pressing part 41 is disposed.

The rod member 50 is installed at the main body 10 to be linearly movable, and disposed at a rear side of the needle 30 to be on a line with the needle 30, while being spaced from the needle 30.

The rod member 50 primarily moves the follicle 90 in the follicle supply pipe 35 moved in the main body 10 by the follicle supply part 20 and the transfer part 40 to an inner side of the needle 30, and then secondarily moves the follicle 90 disposed in the inner side of the needle 30 to an outside, i.e., toward a scalp 100.

To this end, a diameter of the rod member 50 is formed smaller than inner diameters of the follicle supply pipe 35 and the needle 30.

The rod driving part 60 is installed in the main body 10 to move the rod member 50 linearly.

Hereinafter, an operating method and procedure of the present invention having the above-described structure will be described.

First, in a state in which the needle 30 is inserted into the scalp 100, when a separate operation button is pushed, the follicle supply pipe 35 is located at the discharging port 25a by a rotation of the rotating member 22, as illustrated in FIGS. 3A and 4A.

At this time, the follicle supply pipe 35 is supported downward by the lower pressing part 42.

In this state, when the upper pressing part 41 is moved down by the up and down driving part 43, the follicle supply pipe 35 is moved down while being inserted between the upper pressing part 41 and the lower pressing part 42, as illustrated in FIGS. 3B and 4B. At this time, the lower spring 44 is compressed.

The follicle supply pipe 35 moved down by the transfer part 40 is disposed in a straight line between the rod member 50 and the needle 30.

Then, as illustrated in FIG. 4C, the rod member 50 is moved forward by the rod driving part 60 to move forward the follicle 90 in the follicle supply pipe 35.

And as illustrated in FIG. 4D, the follicle 90 passes through the follicle supply pipe 35 and the needle 30, and then is inserted into the scalp 100.

As described above, when the follicle 90 is inserted into the scalp 100, the rod member 50 is moved backward, as illustrated in FIG. 4E, and the transfer part 40 moves up a waste follicle supply pipe 36, in which the follicle 90 is used and thus does not exist, to the follicle supply part 20 as illustrated in FIGS. 3C and 4F.

At this time, the lower pressing part 42 is moved up by an elastic restoring force of the compressed lower spring 44.

Then, as illustrated in FIG. 3D, the rotation driving part rotates the rotating member 22 so that a new follicle supply pipe 35 is disposed at the discharging port 25a.

Meanwhile, when all of the follicles 90 in the follicle supply part 20 are used, the follicle supply part 20 may be replaced in a cartridge manner.

Through the above-described operation procedure, when one of the follicles 90 is implanted into the scalp 100 by the rod member 50, the follicle supply part 20 and the transfer part 40 automatically supply the new follicle 90 in front of the rod member 50, and thus the follicle 90 can be rapidly and easily implanted into the scalp 100.

### Second Embodiment

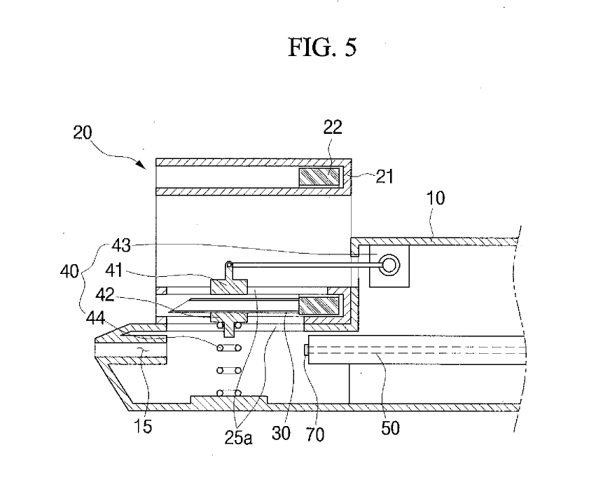

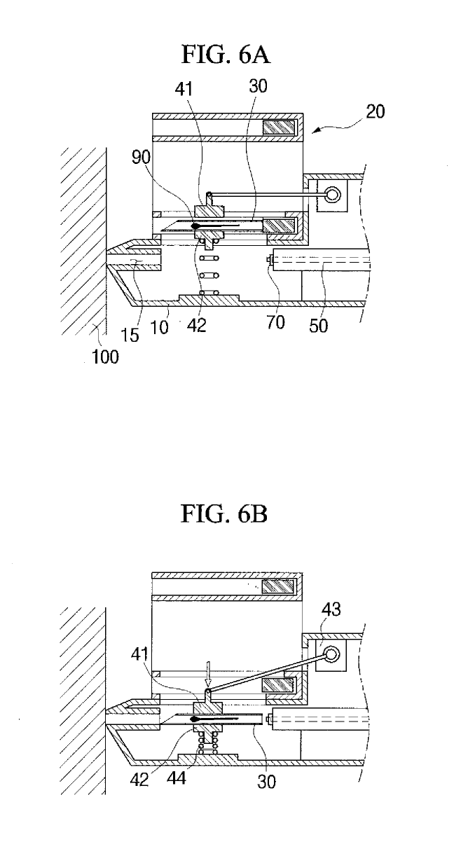

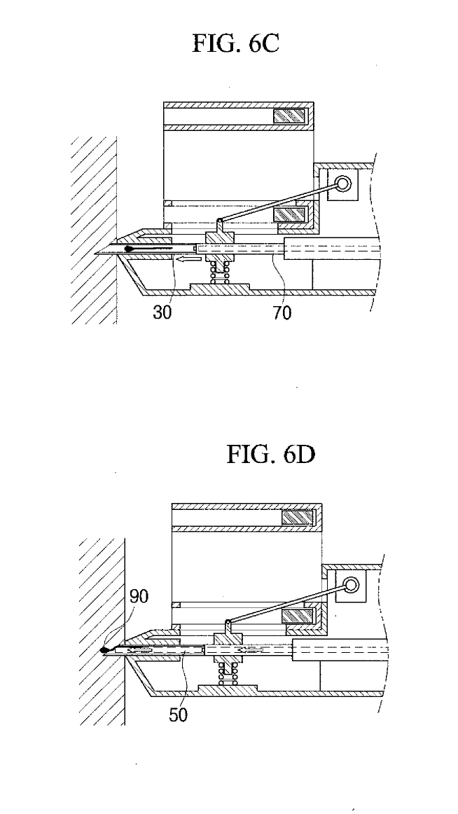

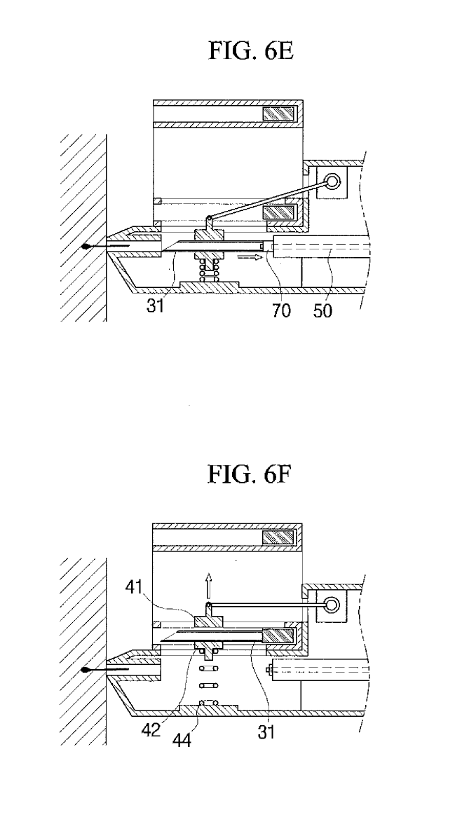

FIG. 5 is a structural view of a follicle supply part and a transfer part of an injector for hair implant according to a second embodiment of the present invention, and FIGS. 6A to 6F is a view illustrating an operation procedure of the injector for hair implant according to the second embodiment of the present invention.

As illustrated in FIGS. 5 and 6, the injector for hair implant according to the present invention includes a main body 10, a follicle supply part 20, a needle 30, a transfer part 40, a rod member 50, and a rod driving part 60.

The second embodiment is different from the first embodiment in the follicle supply pipe 35 and the needle 30. Description will be provided focusing thereon.

In the second embodiment, the follicle supply pipe 35 disposed at the follicle supply part 20 is configured with the needle 30.

That is, the needle 30 serves as the follicle supply pipe 35, and each follicle 90 is inserted into a plurality of needles 30.

Therefore, in the second embodiment, a separate needle is not coupled to the front side of the main body 10, and a passage 15 through which the needle 30 is moved forward and backward is formed at the main body 10.

The second embodiment further includes a needle moving part 70 which moves the needle 30 supplied from the follicle supply part 20 to the front side of the main body 10.

The needle moving part 70 grasps and moves linearly the needle 30 supplied from the follicle supply part 20 and disposed in front of the rod member 50.

Therefore, the needle 30 is disposed at the front side of the main body 10 by the needle moving part 70.

At this time, the needle moving part 70 is coupled to or in close contact with the needle 30 at different positions from each other so as to prevent interference with the upper pressing part 41 and the lower pressing part 42, when the needle moving part 70 is moved.

The rod member 50 which linearly moves the follicle 90 is installed at an inner side of the needle moving part 70 to be linearly movable.

Since other configuration is the same as that of the first embodiment, detailed description thereof will be omitted.

Hereinafter, an operating method and procedure of the second embodiment having the above-described structure will be described.

As illustrated in FIGS. 6A and 6B, disposing of the needle 30 in the follicle supply part 20 in front of the rod member 50 is the same as the disposing of the follicle supply pipe 35 in front of the rod member 50 in the first embodiment, and thus description thereof will be omitted.

As illustrated in FIG. 6B, the needle 30 moved down by the transfer part 40 is disposed to be on a line with the rod member 50.

Then, as illustrated in FIG. 6C, the needle moving part 70 grasps, moves forward and then inserts the needle 30 into the scalp 100.

At this time, the rod member 50 is moved along with the needle moving part 70, while being installed at the inner side of the needle moving part 70.

In this state, as illustrated in FIG. 6D, as the rod member 50 is moved forward by the rod driving part 60, the follicle 90 in the needle 30 is inserted into the scalp 100.

When the follicle 90 is inserted into the scalp 100, the rod member 50 and the needle 30 are moved backward as illustrated in FIG. 6E, and the transfer part 40 moves up a waste needle 31, in which the follicle 90 is used and thus does not exist, to the follicle supply part 20, as illustrated in FIG. 6F.

At this time, the lower pressing part 42 is moved up by the elastic restoring force of the compressed lower spring 44.

Disposing of the new needle 30 at the discharging port 25a by the follicle supply part 20 is the same as the disposing of the follicle supply pipe 35 at the discharging port 25a in the first embodiment, and thus description thereof will be omitted.

Through above-described operation procedure, the new needle 30 is used whenever the follicle 90 is implanted in the scalp 100, and thus the second embodiment is more sanitary than the first embodiment.

### Third Embodiment

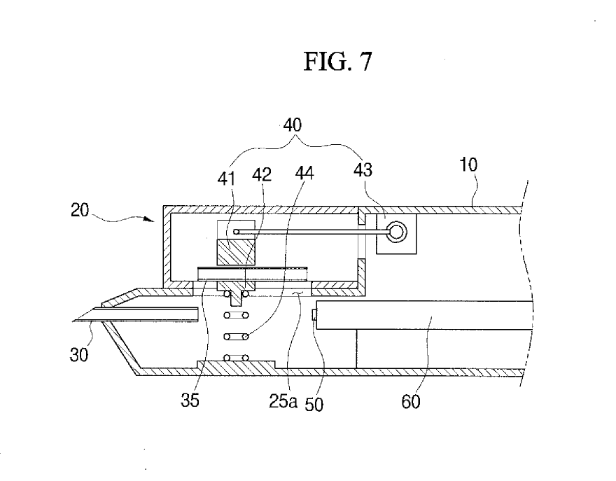

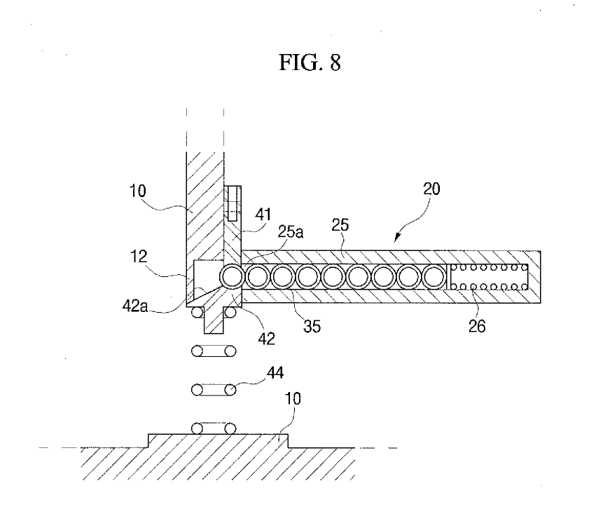

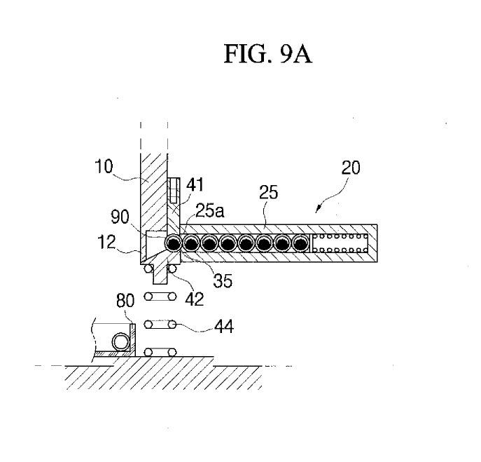

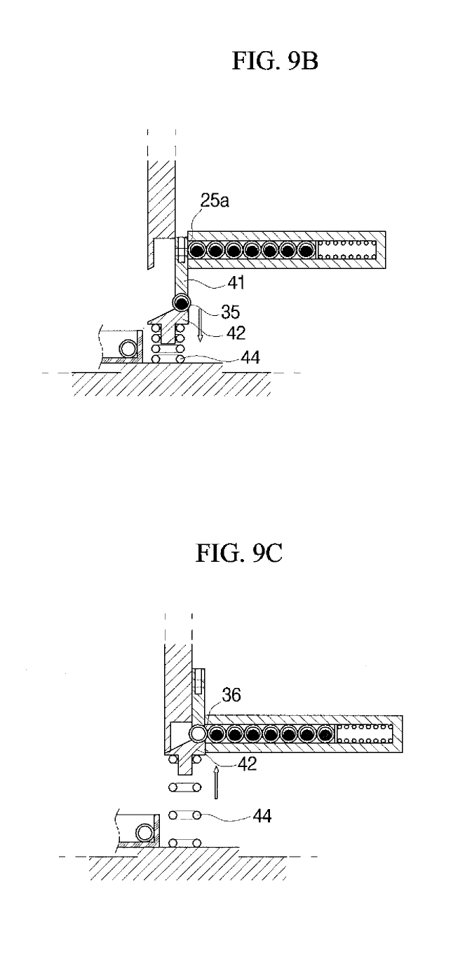

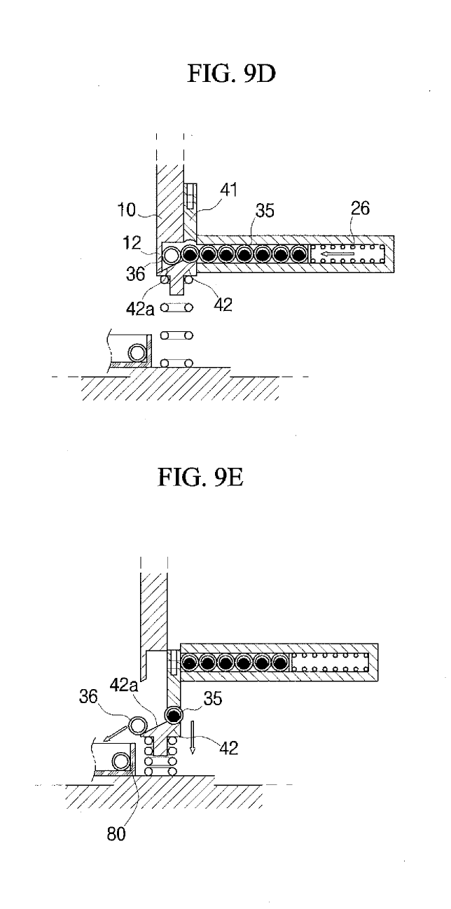

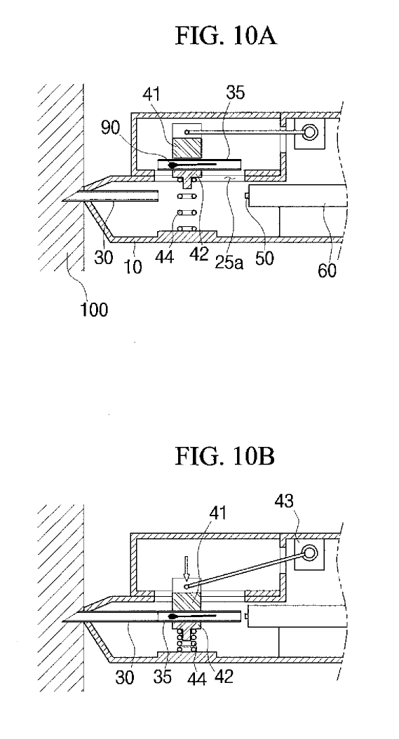

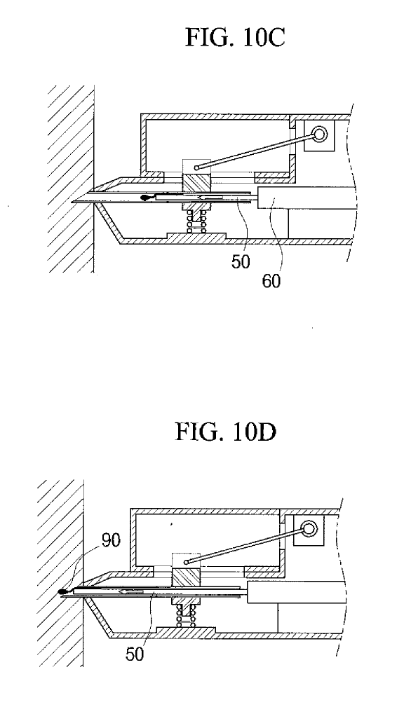

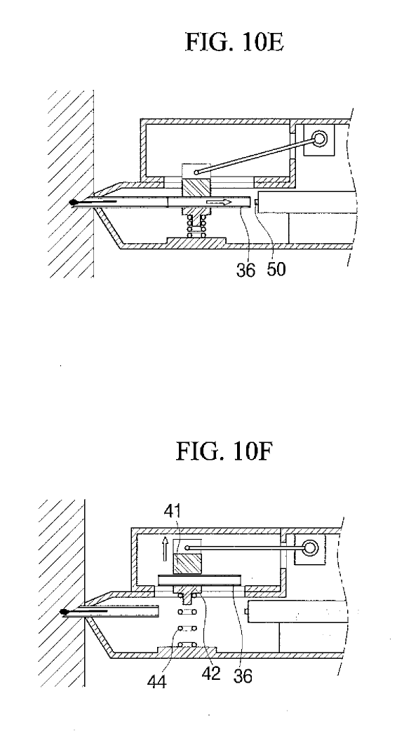

FIG. 7 is a structural view of an injector for hair implant according to a third embodiment of the present invention, FIG. 8 is a structural view of a follicle supply part and a transfer part of the injector for hair implant according to the third embodiment of the present invention, FIGS. 9A to 9E is a view illustrating an operation procedure of the follicle supply part and the transfer part of the injector for hair implant according to the third embodiment of the present invention, and FIGS. 10A to 10F is a view illustrating the operation procedure of the injector for hair implant according to the third embodiment of the present invention.

As illustrated in FIGS. 7 to 10, the injector for hair implant according to the third embodiment of the present invention includes a main body 10, a follicle supply part 20, a needle 30, a transfer part 40, a rod member 50, and a rod driving part 60.

The third embodiment is different from the first embodiment in the follicle supply part 20 and the transfer part 40. Description will be provided focusing thereon.

The follicle supply part 20 includes a housing 25 and a push member 26.

The housing 25 has a long rectangular parallelepiped shape, and the plurality of the follicle supply pipes 35 are disposed therein in a row, and the discharging port 25a is formed at one end thereof.

The housing 25 may be detachably installed at the main body 10.

The push member 26 is installed in the housing 25, and serves to push the follicle supply pipe 35 toward the discharging port 25a.

The push member 26 may have various structures. In the embodiment, the push member 26 is configured with a pressing spring.

Therefore, the follicle supply pipe 35 disposed in a row is pushed and moved toward the discharging port 25a by an elastic restoring force, i.e., a pushing force of the pressing spring 26.

The transfer part 40 moves the follicle supply pipe 35 discharged through the discharging port 25a by the push member, i.e., the pressing spring 26 to the front side of the rod member 50.

The transfer part 40 includes an upper pressing part 41, a lower pressing part 42, and an up and down driving part 43.

The upper pressing part 41 is disposed at a front upper side of the discharging port 25a, and a concave groove corresponding to a shape of the follicle supply pipe 35 is formed at a lower surface thereof.

The lower pressing part 42 is disposed at a front lower side of the discharging port 25a, and a concave groove corresponding to the shape of the follicle supply pipe 35 is formed at an upper surface thereof.

The up and down driving part 43 serves to move the upper pressing part 41 and the lower pressing part 42 up and down, and may have various conventional structures.

Due to the transfer part 40, the follicle supply pipe 35 may be separated from the follicle supply part 20 by a downward movement of the upper pressing part 41 and the lower pressing part 42 by the up and down driving part 43, while being discharged through the discharging port 25a and disposed between the upper pressing part 41 and the lower pressing part 42, and then may be disposed in front of the rod member 50.

Of course, the transfer part 40 may move the follicle supply pipe 35 downward and then upward again.

A lower spring 44 is installed at a lower surface of the lower pressing part 42 to elastically support the lower pressing part 42 in an upward direction in which the upper pressing part 41 is disposed.

Also, when the upper pressing part 41 is moved down, the discharging port 25a is closed by a side surface of the upper pressing part 41.

Meanwhile, the transfer part 40 moves a waste follicle supply pipe 36, in which the follicle 90 is used, in a lateral direction of the discharging port 25a, and the push member 26 pushes the follicle supply pipe 35 arranged in the housing 25 toward the discharging port 25a, and thus separates the waste follicle supply pipe 36 from the transfer part 40.

At this time, a stopper 12 by which the waste follicle supply pipe 36 disposed between the upper pressing part 41 and the lower pressing part 42 and horizontally moved by the elastic restoring force of the pressing spring 26 is caught and stopped is formed at the main body 10.

The lower pressing part 42 supports the waste follicle supply pipe 36 caught and stopped by the stopper 12.

And when the lower pressing part 42 is moved down, the waste follicle supply pipe 36 stopped by the stopper 12 is moved down along with the lower pressing part 42, and then moved to a separate collecting part 80.

An inclined surface 42a inclined in a direction opposite to a supply direction of the new follicle supply pipe 35 is formed at a portion of an upper surface of the lower pressing part 42 in which the waste follicle supply pipe 36 is disposed.

Therefore, when the lower pressing part 42 is moved down, the waste follicle supply pipe 36 disposed at an upper portion of the inclined surface 42a may be easily moved along the inclined surface 42a to the collecting part 80.

Hereinafter, an operating method and procedure of the third embodiment having the above-described structure will be described.

As illustrated in FIGS. 9A and 10A, in an initial state, the follicle supply pipe 35 discharged from the discharging port 25a is disposed between the upper pressing part 41 and the lower pressing part 42.

When an operator pushes a separate operation button while the needle 30 is inserted into the scalp 100, the upper pressing part 41 is moved down by the up and down driving part 43, and the follicle supply pipe 35 is moved down while being disposed between the upper pressing part 41 and the lower pressing part 42, and at this time, the lower spring 44 is compressed, as illustrated in FIGS. 9B and 10B.

The follicle supply pipe 35 moved down by the transfer part 40 is disposed in a row between the rod member 50 and the needle 30.

At this time, the discharging port 25a formed at the housing 25 is closed by the side surface of the upper pressing part 41 due to the downward movement of the upper pressing part 41.

Then, as illustrated in FIG. 10C, the rod member 50 is moved forward by the rod driving part 60, and thus the follicle 90 in the follicle supply pipe 35 is also moved forward.

As illustrated in FIG. 10d, the follicle 90 passes through the follicle supply pipe 35 and the needle 30, and then is inserted into the scalp 100.

When the follicle 90 is inserted into the scalp 100, the rod member 50 is moved backward, as illustrated in FIG. 10E, and the transfer part 40 moves up a waste follicle supply pipe 36, in which the follicle 90 is used and thus does not exist, in the lateral direction of the discharging port 25a, as illustrated in FIGS. 9C and 10F.

At this time, the lower pressing part 42 is moved up by the elastic restoring force of the compressed lower spring 44.

As the upper pressing part 41 is moved up, the discharging port 25a is opened, and the follicle supply pipe 35 pushed toward the discharging port 25a by the push member 26 pushes and separates the waste follicle supply pipe 36 disposed between the upper pressing part 41 and the lower pressing part 42 from the transfer part 40, and then is disposed thereto.

At this time, the waste follicle supply pipe 36 separated from the transfer part 40 is horizontally moved and then caught and stopped by the stopper 12.

Therefore, the follicle supply pipes 35 in the housing 25 are not discharged to the discharging port 25a any more, and are maintained in this state.

A lower portion of the waste follicle supply pipe 36 caught and stopped by the stopper 12 is supported by the lower pressing part 42.

In this state, when the operation button is pushed again, the transfer part 40 is moved down, and the waste follicle supply pipe 36 caught and stopped by the stopper 12 is moved down along with the lower pressing part 42 and moved and collected to the collecting part 80 along the inclined surface 42a, as illustrated in FIG. 9E.

When all of the follicles 90 in the follicle supply part 20 are used, the follicle supply part 20 may be replaced in a cartridge manner.

Through the above-described operation procedure, when one of the follicles 90 is implanted into the scalp 100 by the rod member 50, the follicle supply part 20 and the transfer part 40 automatically supply the new follicle 90 in front of the rod member 50, and thus the follicle 90 may be rapidly and easily implanted into the scalp 100.

### Fourth Embodiment

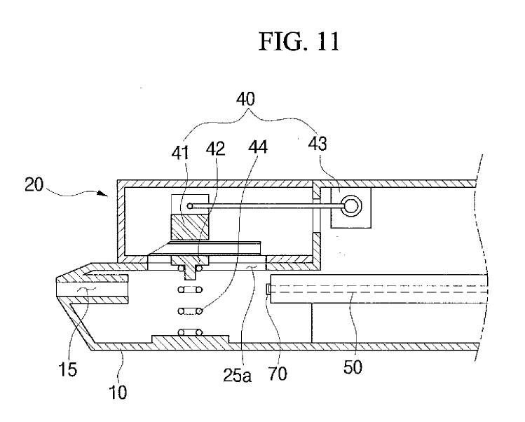

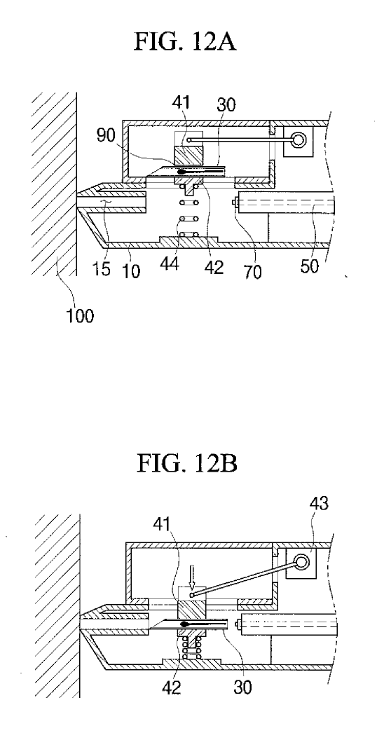

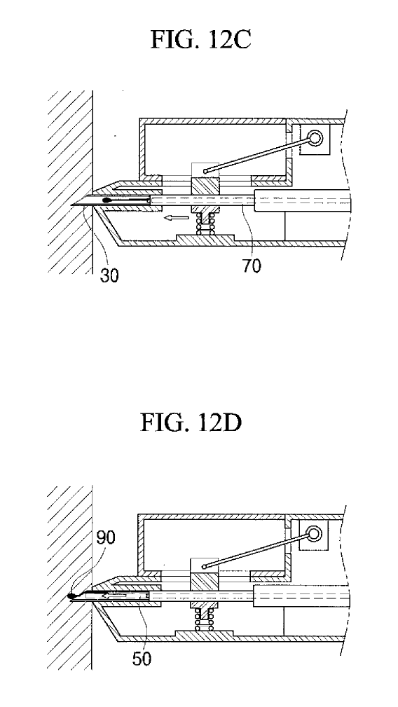

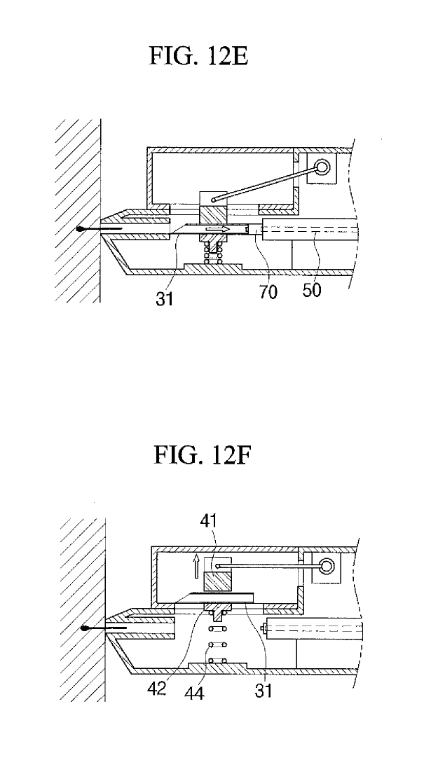

FIG. 11 is a structural view of an injector for hair implant according to a fourth embodiment of the present invention, and FIGS. 12A to 12F is a view illustrating an operation procedure of the injector for hair implant according to the fourth embodiment of the present invention.

As illustrated in FIGS. 11 and 12, the injector for hair implant according to the fourth embodiment includes a main body 10, a follicle supply part 20, a needle 30, a transfer part 40, a rod member 50, and a rod driving part 60.

The fourth embodiment is different from the third embodiment in the follicle supply pipe 35 and the needle 30. Description will be provided focusing thereon.

In the fourth embodiment, the follicle supply pipe 35 disposed at the follicle supply part 20 is configured with the needle 30.

That is, the needle 30 serves as the follicle supply pipe 35, and each follicle 90 is inserted into a plurality of needles 30.

Therefore, in the fourth embodiment, a separate needle is not coupled to the front side of the main body 10, and a passage 15 through which the needle 30 is moved forward and backward is formed at the main body 10.

As described above, the thing that the follicle supply pipe is configured with the needle 30 and linearly moved is the same as that in the second embodiment.

The fourth embodiment further includes a needle moving part 70 which moves the needle 30 supplied from the follicle supply part 20 to the front side of the main body 10.

The needle moving part 70 grasps and moves linearly the needle 30 supplied from the follicle supply part 20 and disposed in front of the rod member 50.

Therefore, the needle 30 is disposed at the front side of the main body 10 by the needle moving part 70.

And the rod member 50 which linearly moves the follicle 90 is installed at an inner side of the needle moving part 70 to be linearly movable.

Since other configuration is the same as that of the third embodiment, detailed description thereof will be omitted.

Hereinafter, an operating method and procedure of the fourth embodiment having the above-described structure will be described.

As illustrated in FIGS. 12A and 12B, disposing of the needle 30 in the follicle supply part 20 in front of the rod member 50 is the same as the disposing of the follicle supply pipe 35 in front of the rod member 50 in the third embodiment, and thus description thereof will be omitted.

As illustrated in FIG. 12B, the needle 30 moved down by the transfer part 40 is disposed to be on a line with the rod member 50.

Then, as illustrated in FIG. 12C, the needle moving part 70 grasps, moves forward and then inserts the needle 30 into the scalp 100.

At this time, the rod member 50 is moved along with the needle moving part 70, while being installed at the inner side of the needle moving part 70.

In this state, as illustrated in FIG. 12D, as the rod member 50 is moved forward by the rod driving part 60, the follicle 90 in the needle 30 is inserted into the scalp 100.

When the follicle 90 is inserted into the scalp 100, the rod member 50 and the needle 30 are moved backward as illustrated in FIG. 12E, and the transfer part 40 moves up the waste needle 31, in which the follicle 90 is used and thus does not exist, as illustrated in FIG. 12F.

At this time, the lower pressing part 42 is moved up by the elastic restoring force of the compressed lower spring 44.

And, the things that the new needle 30 is supplied from the discharging port 25a by the follicle supply part 20, and the waste needle 31 is collected to the collecting part 80 are the same as those of the third embodiment in which the follicle supply pipe 35 is supplied from the discharging port 25a, and a waste follicle supply pipe 36 is collected to the collecting part 80, and thus description thereof will be omitted.

Through above-described operation procedure, the new needle 30 is used whenever the follicle 90 is implanted in the scalp 100, and thus the fourth embodiment is more sanitary than the third embodiment.

According to the injector for hair implant with the automatic follicle supply function of the present invention, as described above, the follicles can be automatically and continuously supplied, and thus can be rapidly and easily implanted in the scalp.

Also, the follicle implant time can be reduced by only replacing the cartridge type follicle supply part in which the plurality of follicles are disposed.

It will be apparent to those skilled in the art that various modifications can be made to the above-described exemplary embodiments of the present invention without departing from the spirit or scope of the invention. Thus, it is intended that the present invention covers all such modifications provided they come within the scope of the appended claims and their equivalents.

What is claimed is:

1. An injector for hair implant with an automatic follicle supply function, comprising:
a main body;
a follicle supply part coupled with the main body to supply a plurality of follicles;
a needle disposed at a front side of the main body;
a rod member installed at the main body to move forward the follicle supplied through the follicle supply part and disposed in the needle;
a transfer part configured to move the follicle disposed at the follicle supply part to a front side of the rod member; and
a rod driving part installed at the main body to linearly move the rod member,
wherein the follicle supply part and the transfer part automatically supply a new follicle in front of the rod member, when one of the follicles is implanted into a scalp by the rod member.

2. The injector of claim 1, wherein a plurality of follicle supply pipes are disposed in the follicle supply part to be supplied to the main body, and the follicles are disposed in the follicle supply pipe, respectively.

3. The injector of claim 2, wherein the follicle supply part comprises a fixed member formed in a cylindrical shape of which an outer circumferential surface is disposed in an up and down direction; a rotating member rotatably disposed at an inner side of the fixed member; and a rotation driving part configured to rotate the rotating member, and
the plurality of follicle supply pipes are installed at the rotating member to revolve around a rotating center point of the rotating member, when the rotating member is rotated.

4. The injector of claim 3, wherein a discharging port configured to discharge the follicle supply pipe installed at the rotating member to an outside of the follicle supply part is formed at the fixed member in an up and down direction.

5. The injector of claim 4, wherein the transfer part comprises an upper pressing part disposed above the discharging port; a lower pressing part disposed under the discharging port; and an up and down driving part configured to move upward and downward the upper pressing part and the lower pressing part, and
the follicle supply pipe disposed at the discharging port is separated from the follicle supply part by a downward movement of the upper and lower pressing parts due to the up and down driving part, and then disposed in front of the rod member.

6. The injector of claim 5, wherein the transfer part moves again a waste follicle supply pipe in which the follicle is used to the follicle supply part, and the rotation driving part rotates the rotating member and disposes a new follicle supply pipe at the discharging port.

7. The injector of claim 5, wherein a lower spring is installed at a lower surface of the lower pressing part to elastically support the lower pressing part in an upward direction in which the upper pressing part is disposed.

8. The injector of claim 2, wherein the follicle supply part comprises a housing having the plurality of the follicle supply pipes disposed therein in a row, and a discharging port formed at one end thereof; and a push member installed in the housing to push the follicle supply pipe toward the discharging port, and
the transfer part moves the follicle supply pipe discharged through the discharging port by the push member to the front side of the rod member.

9. The injector of claim 8, wherein the transfer part comprises an upper pressing part disposed at a front upper side of the discharging port; a lower pressing part disposed at a front lower side of the discharging port; and an up and down driving part configured to move upward and downward the upper pressing part and the lower pressing part, and
the follicle supply pipe disposed between the upper pressing part and the lower pressing part is disposed in front of the rod member by a downward movement of the upper and lower pressing parts due to the up and down driving part.

10. The injector of claim 9, wherein a lower spring is installed at a lower surface of the lower pressing part to elastically support the lower pressing part in an upward direction in which the upper pressing part is disposed.

11. The injector of claim 9, wherein the discharging port is closed when the upper pressing part is moved down.

12. The injector of claim 9, wherein the transfer part moves a waste follicle supply pipe, in which the follicle is used, in a lateral direction of the discharging port, and
the push member pushes the follicle supply pipe disposed in the housing toward the discharging port and thus separates the waste follicle supply pipe from the transfer part.

13. The injector of claim 11, wherein the push member is a pressing spring, and a stopper by which the waste follicle supply pipe disposed between the upper pressing part and the lower pressing part and horizontally moved by the elastic restoring force of the pressing spring is stopped is formed at the main body, and the lower pressing part supports the waste follicle supply pipe caught and stopped by the stopper, and when the lower pressing part is moved down, the waste follicle supply pipe caught and stopped by the stopper is moved down along with the lower pressing part and then moved to a collecting part.

14. The injector of claim 13, wherein an inclined surface inclined in a direction opposite to a supply direction of the new follicle supply pipe is formed at a portion of an upper surface of the lower pressing part in which the waste follicle supply pipe is disposed, and
when the lower pressing part is moved down, the waste follicle supply pipe disposed at an upper portion of the inclined surface is moved to the collecting part along the inclined surface.

15. The injector of claim 2, wherein the needle is fixed to a front side of the main body, and disposed to be on a line with the rod member, and the follicle supply pipe supplied to the main body by the transfer part is disposed in a row between the rod member and the needle.

16. The injector of claim 2, wherein the follicle supply pipe is configured with the needle, and a needle moving part which linearly moves the needle supplied from the follicle supply part and disposed in front of the rod member is installed at the main body, and the needle is disposed in front of the main body by the needle moving part.

17. The injector of claim 16, wherein the rod member is installed at an inner side of the needle moving part to be linearly movable.
